(19) United States
(12) Patent Application Publication
(10) Pub. No.: US 2017/0153235 A1
(43) Pub. Date: Jun. 1, 2017

(54) APTAMER FOR SPECIFICALLY DETECTING PATULIN AND PATULIN DETECTION METHOD USING THE SAME

(71) Applicant: GWANGJU INSTITUTE OF SCIENCE AND TECHNOLOGY, Gwangju (KR)

(72) Inventors: Min-Gon KIM, Gwangju (KR); Hyo-young MUN, Gwangju (KR); Min-Jin KIM, Gwangju (KR); Won-Bo SHIM, Jinju-si (KR)

(21) Appl. No.: 15/349,020

(22) Filed: Nov. 11, 2016

(30) Foreign Application Priority Data
Nov. 13, 2015 (KR) 10-2015-0159498

(51) Int. Cl. G01N 33/569

(52) U.S. Cl. CPC G01N 33/56961; G01N 2333/38; G01N 2333/385

(57) ABSTRACT

An aptamer for specifically detecting patulin and a method for detecting patulin using the same. The aptamer for specifically detecting patulin is a single-stranded DNA aptamer serving as a bioreceptor capable of effectively detecting patulin. Since such an aptamer for specifically detecting patulin is capable of specifically binding to patulin which is a mycotoxin having a simple chemical structure and a low molecular weight, it can be effectively employed as a bioreceptor in various bioassays for specifically detecting patulin. The aptamer for specifically detecting patulin can achieve more effective detection of patulin in apples or apple juice.

### CROSS REFERENCE TO RELATED APPLICATION

This application claims the benefit of Korean Patent Application No. 10-2015-0159498, filed on Nov. 13, 2015, entitled “APTAMER FOR SPECIFICALLY DETECTING PATULIN AND PATULIN DETECTION METHOD USING THE SAME”, which is hereby incorporated by reference in its entirety into this application.

### BACKGROUND

1. Technical Field

The present invention relates to an aptamer for specifically detecting patulin and a method for detecting patulin using the same.

2. Description of the Related Art

Patulin is a mycotoxin produced by fungal species of the genera Penicillium, Aspergillus, and Byssochlamys and found in rotten fruits, such as apples, pears, grapes, peaches, and the like and drinks made from such rotten fruits, specifically often found in apples and apple juice.

However, it is known that patulin generally adversely affects nervous tissues and digestive organs and has toxic properties such as DNA damage, immune suppression activity and the like. Particularly, patulin is known to cause diseases fatal to infants. For these reasons, the EU and FDA have set an upper limit of 50 μg/ml for patulin concentrations in apple juice. Accordingly, preliminary detection and identification of whether patulin is contained in apples or apple juice and the like and determination of patulin concentration are important concerns.

Nevertheless, up to the present time, only typical methods such as HPLC (high performance liquid chromatography), GC (gas chromatography), LC/MS (liquid chromatography/mass spectroscopy), and GC/MS have been employed in detection of patulin. However, such methods have disadvantages in that they require complex procedures for preparing test specimens, expensive analytic devices and experienced researchers, and take a long time to detect patulin.

In order to resolve such disadvantages, bioassay methods which can rapidly detect patulin and are convenient to employ have taken attention. Representative examples include an enzyme-linked immunosorbent assay (ELISA) method and a lateral flow immunoassay (LFA) method. With such methods, it is possible to detect Ochratoxin A, which is a representative mycotoxin, using bioreceptors such as antibodies and aptamers specific to Ochratoxin A. However, although the immunoassay method and the lateral flow immunoassay method as such bioassays are convenient in detection of mycotoxins such as Ochratoxin A or Aflatoxin B1, such methods have a problem in that they cannot easily detect patulin. That is, since patulin is a mycotoxin having a simple chemical formula and a small molecular weight as compared with other toxins produced from molds (other mycotoxins have 1.5-3 fold higher molecular weights compared to patulin), bioreceptors capable of specifically detecting patulin have not been developed in the art, thereby hindering application of the immunoassay method or the lateral flow immunoassay method as such bioassay methods.

As a bioreceptor, an aptamer is a single-stranded DNA or RNA that can specifically bind to a specific target. Such an aptamer exhibits good thermal stability to antibodies, can be easily synthesized, and can easily bind to a variety of chemical substances. However, despite these advantages, there is currently a lack of effort to develop an aptamer as a bioreceptor that can specifically detect patulin.

### BRIEF SUMMARY

The present invention has been conceived to solve such problems in the art and it is an aspect of the present invention to provide an aptamer as a bioreceptor capable of specifically detecting patulin as a mycotoxin having a simple chemical structure and a small molecular weight.

It is another aspect of the present invention to provide a method and kit for effectively detecting patulin using the aptamer.

In accordance with one aspect of the present invention, there is provided an aptamer for specifically detecting patulin, which has a nucleotide sequence selected from the group consisting of SEQ ID NO: 1 to SEQ ID NO: 8.

In accordance with another aspect of the present invention, there is provided a method for detecting patulin, which includes: 1) immobilizing a fluorescent material or a fluorescent quencher onto an aptamer for specifically detecting patulin, the aptamer having a nucleotide sequence selected from the group consisting of SEQ ID NO: 1 to SEQ ID NO: 8; 2) adding the aptamer to which the fluorescent material or the fluorescent quencher is immobilized to a patulin-containing sample; and 3) selecting an aptamer specifically bound to patulin.

In accordance with a further aspect of the present invention, there is provided a kit for detecting patulin including a patulin detector, wherein the patulin detector detects patulin by reacting a patulin specific aptamer with a patulin-containing sample and selecting an aptamer specifically bound to patulin, and the patulin specific aptamer includes an immobilized fluorescent material or a fluorescent quencher and has a nucleotide sequence selected from the group consisting of SEQ ID NO: 1 to SEQ ID NO: 8.

According to the present invention, the aptamer for specifically detecting patulin is a single-stranded DNA aptamer as a bioreceptor capable of effectively detecting patulin. Since such an aptamer for specifically detecting patulin is capable of specifically binding to patulin which is a mycotoxin having a simple chemical structure and a small molecular weight, it can be effectively employed as a bioreceptor in various bioassays for specifically detecting patulin. Such an aptamer for specifically detecting patulin can achieve more effective detection of patulin in apples or apple juice.

### BRIEF DESCRIPTION OF THE DRAWINGS

The above and other aspects, features, and advantages of the invention will become apparent from the detailed description of the following embodiments in conjunction with the accompanying drawings, in which:

FIG. 1 is a diagram of graphene oxide-SELEX procedures for developing an aptamer for specifically detecting patulin in Examples;

FIG. 2 shows a graph depicting a recovery rate of each step when graphene oxide-SELEX procedures are performed according to the diagram of FIG. 1;

FIGS. 3a to 3h shows schematic views of predicted secondary structures of eight aptamer sequences developed using an m-fold program in Examples;

FIG. 4 is a diagram of an experiment for determining specific binding between a patulin aptamer and patulin using an attenuation effect of a patulin-specific aptamer developed in Examples and graphene oxide;

FIG. 5 shows graphs depicting results of specific binding between a patulin aptamer and patulin in a buffer according to the diagram of FIG. 4;

FIG. 6 shows graphs depicting results of specific binding between a patulin aptamer and patulin in apple juice as an actual sample according to the diagram of FIG. 4; and

FIG. 7a and FIG. 7b shows graphs depicting results of specific binding between eight patulin aptamers and patulin in a buffer according to the diagram of FIG. 4.

### DETAILED DESCRIPTION

Hereinafter, exemplary embodiments of the present invention will be described in detail.

As a result of the present inventors' earnest study aimed at developing a single-stranded DNA aptamer for effectively detecting patulin, the present inventors discovered an aptamer for specifically detecting patulin and a method for detecting patulin using the same.

In accordance with one aspect of the present invention, an aptamer for specifically detecting patulin has a nucleotide sequence selected from the group consisting of SEQ ID NO: 1 to SEQ ID NO: 8.

The aptamer according to the present invention further includes a forward primer having a nucleotide sequence represented by SEQ ID NO: 9 or a reverse primer having a nucleotide sequence represented by SEQ ID NO: 10.

The single-stranded DNA aptamer according to the present invention can be utilized as a bioreceptor in a bioassay procedure. As a bioreceptor, the aptamer can effectively detect patulin which has a simple chemical structure and a relatively low molecular weight as compared with other mycotoxins. Accordingly, the aptamer according to the present invention has an excellent effect of specifically detecting patulin.

Generally, patulin is represented by Formula 1:

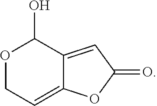 [SMILES: O=C1C=C2C(=CCOC2O)O1]

Since patulin is a mycotoxin having a simple chemical structure and a low molecular weight, it is difficult to develop a bioreceptor for patulin, thereby making it difficult to detect patulin through bioassays. The present invention is aimed at developing and providing an aptamer for solving this problem.

As a method for developing the aptamer according to the present invention, any typical method known in the art may be used without specific limitation. Preferably, the aptamer according to the present invention is developed using a graphene oxide-SELEX method. The graphene oxide-SELEX method refers to a method for determining a nucleotide sequence of a DNA having a high binding force specifically to a specific target, which includes: selecting a DNA having a high binding force specifically to a specific target and amplifying the corresponding DNA in a randomly synthesized random DNA library. Further, the graphene oxide-SELEX method employs graphene oxide in a selection procedure and utilizes strong adsorption of exposed nucleobases in the single-stranded DNA to a surface of graphene oxide. Furthermore, the DNA reacted with a specific target in the graphene oxide-SELEX method is not bound to graphene oxide and thus employed in the following experiment, whereas the DNA un-reacted with a specific target is bound to graphene oxide, which is removed by centrifugation. The present inventors developed an aptamer using the graphene oxide-SELEX method requiring such procedures. Further, lambda exonuclease I is an enzyme having an activity to digest 5′-phosphorylated strand of double-stranded DNA and is used in a process of making a single-stranded DNA from the double-stranded DNA resulting from a polymerase chain reaction during the graphene oxide-SELEX procedure. In order to utilize such an activity, a primer is phosphorylated by immobilizing a phosphate when constructing primer sets. In addition, the aptamer for specifically detecting patulin may be amplified using PCR (polymerase chain reaction) and the like. The single-stranded aptamer before amplification can be converted into double-stranded DNA through amplification. Furthermore, although it is not particularly limited, the double-stranded DNA can be converted again by lambda exonuclease I into single-stranded DNA, to which a fluorescent material or a fluorescent quencher is immobilized. The procedures of constructing a single-stranded aptamer after amplification can be repeated.

In accordance with another aspect of the present invention, a method for detecting patulin employs the aptamer for specifically detecting patulin as set forth above and includes: 1) immobilizing a fluorescent material or a fluorescent quencher onto an aptamer for specifically detecting patulin, which has a nucleotide sequence selected from the group consisting of SEQ ID NO: 1 to SEQ ID NO: 8; 2) adding the aptamer to which the fluorescent material or the fluorescent quencher is immobilized to a patulin-containing sample; and 3) selecting an aptamer specifically bound to patulin.

The aptamer further includes a forward primer having a nucleotide sequence represented by SEQ ID NO: 9, or a reverse primer having a nucleotide sequence represented by SEQ ID NO: 10.

The fluorescent material or the fluorescent quencher is preferably at least one selected from the group consisting of graphene oxide, fluorescein, tetramethylrhodamine, Cy5 (cyanine 5), Cy3 (cyanine 3), and Texas Red.

The fluorescence of the aptamer to which the fluorescent material or the fluorescent quencher is immobilized can be recovered by adding the aptamer to a patulin-containing sample and binding the aptamer to patulin. Namely, a fluorescence signal of fluorescent aptamer quenched by the fluorescent material or the fluorescent quencher can be identified by adding patulin as a target material and binding patulin with the aptamer with a stronger binding force as compared to the fluorescent material or the fluorescent quencher, while releasing the fluorescent material or the fluorescent quencher.

In accordance with a further aspect of the present invention, there is provided a kit for detecting patulin, which includes a patulin detector, wherein the patulin detector detects patulin by reacting a patulin specific aptamer with a patulin-containing sample and selecting an aptamer specifically bound to patulin, and the patulin specific aptamer includes an immobilized fluorescent material or a fluorescent quencher and has a nucleotide sequence selected from the group consisting of SEQ ID NO: 1 to SEQ ID NO: 8.

The aptamer further includes a forward primer having a nucleotide sequence represented by SEQ ID NO: 9, or a reverse primer having a nucleotide sequence represented by SEQ ID NO: 10.

The fluorescent material or the fluorescent quencher is preferably at least one selected from the group consisting of graphene oxide, fluorescein, tetramethylrhodamine, Cy5, Cy3 and Texas Red.

The kit for detecting patulin according to the present invention is capable of detecting patulin by means of the following procedure, without being particularly limited. To be specific, the fluorescence of the aptamer to which the fluorescent material or the fluorescent quencher is immobilized can be recovered by adding the aptamer to a patulin-containing sample and binding the aptamer to patulin. Namely, the fluorescence signal of fluorescent aptamer quenched by the fluorescent material or the fluorescent quencher can be identified by adding patulin as a target material and binding patulin with the aptamer with a stronger binding force as compared to the fluorescent material or the fluorescent quencher, while releasing the fluorescent material or the fluorescent quencher.

Hereinafter, the present invention will be described in more detail with reference to some examples. It should be understood that these examples are provided for illustration only and are not to be construed in any way as limiting the present invention.

### Example

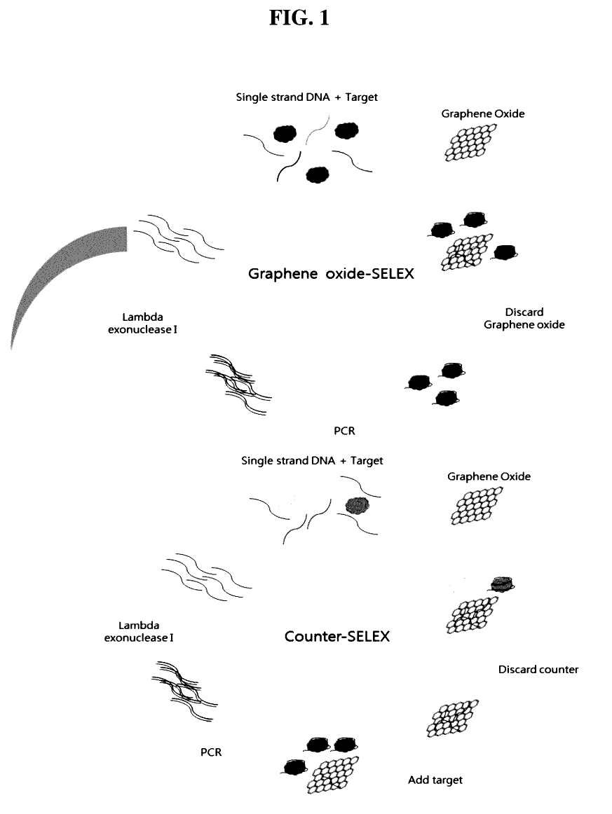

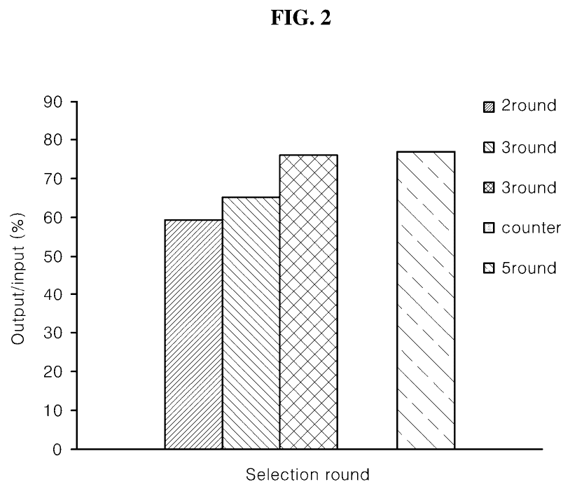

Development of an aptamer according to the present invention for specifically detecting patulin was performed using a graphene oxide-SELEX method (FIG. 1 is a diagram of graphene oxide-SELEX procedures for developing an aptamer for specifically detecting patulin in Examples), which shows an increasing recovery rate in the rounds, as depicted in FIG. 2. The recovery rate can be calculated from a ratio of adsorption strength of residual DNA after each round at a wavelength of 260 nm (output) to adsorption strength of added DNA at a wavelength of 260 nm (input)×100(%). After repeating the procedure of FIG. 1 several times, the recovery rate at round 4 (4 times repeat) was approximately 80%. Subsequently, a procedure to remove a non-specific DNA sequence bound to apple juice was added. Finally, the SELEX procedure for patulin was performed one more time to secure a DNA library specific to patulin.

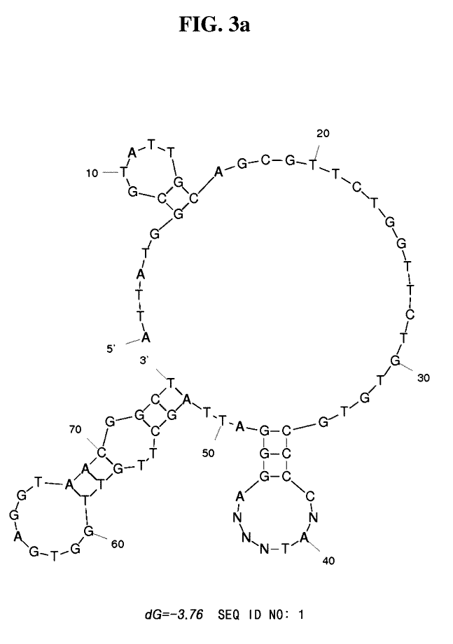

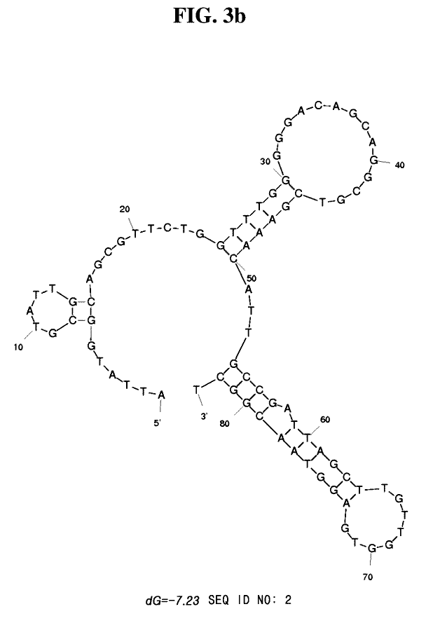

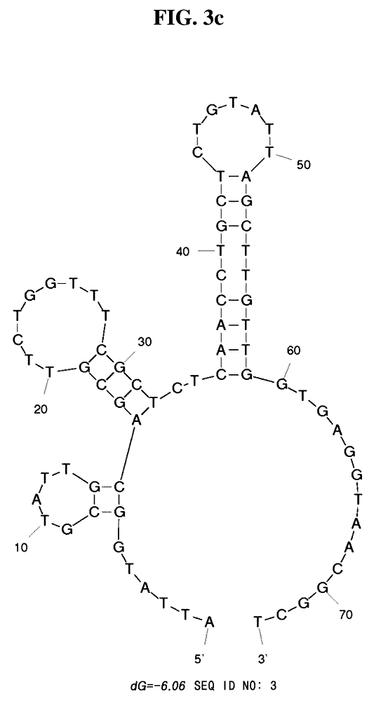

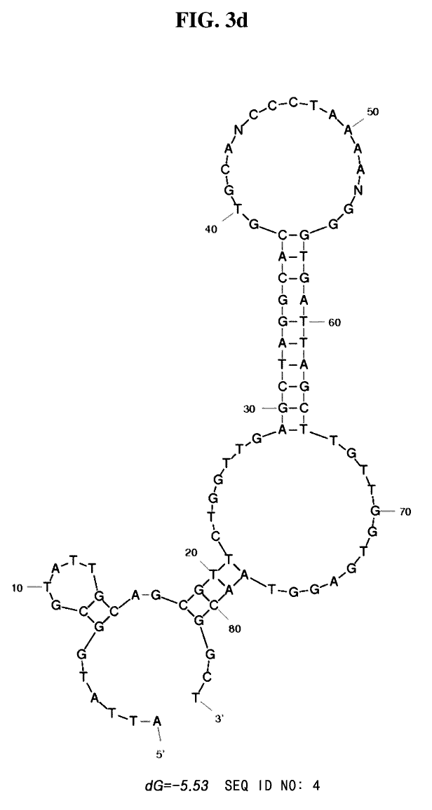

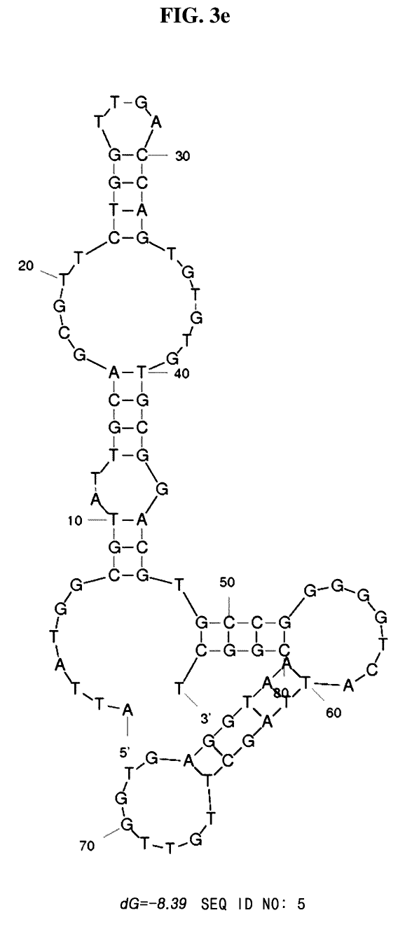

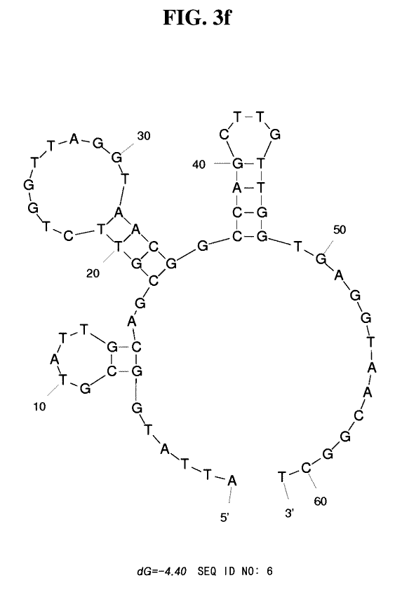

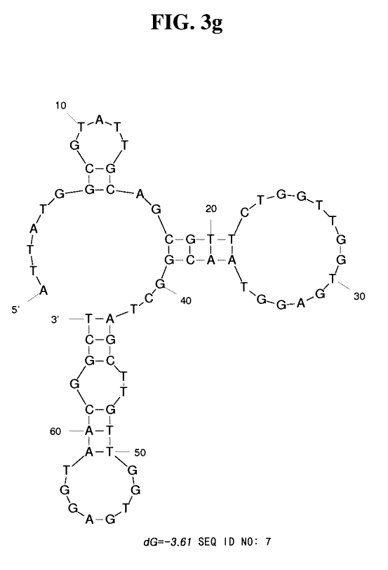

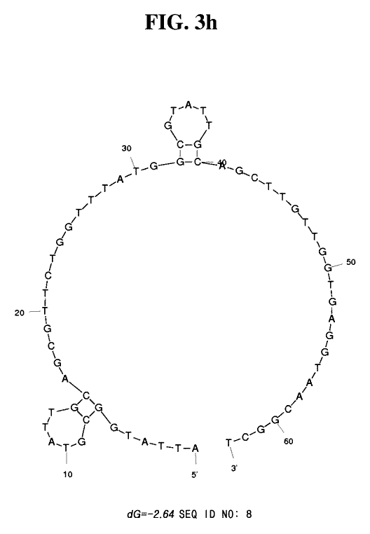

The nucleotide sequence of DNA was analyzed through DNA sequencing. The following Table 1 provides nucleotide sequences for eight aptamers for specifically detecting patulin secured in the above procedures. FIGS. 3a to 3h depict secondary structures of eight patulin-specific aptamers predicted using an m-fold program.

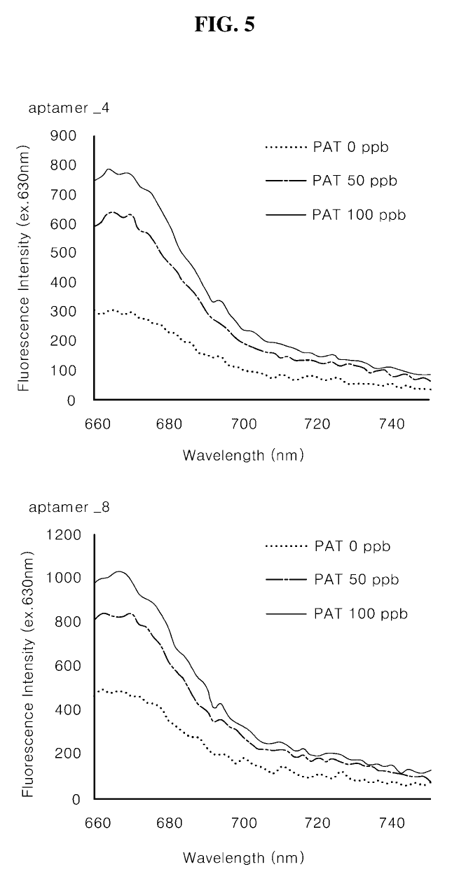

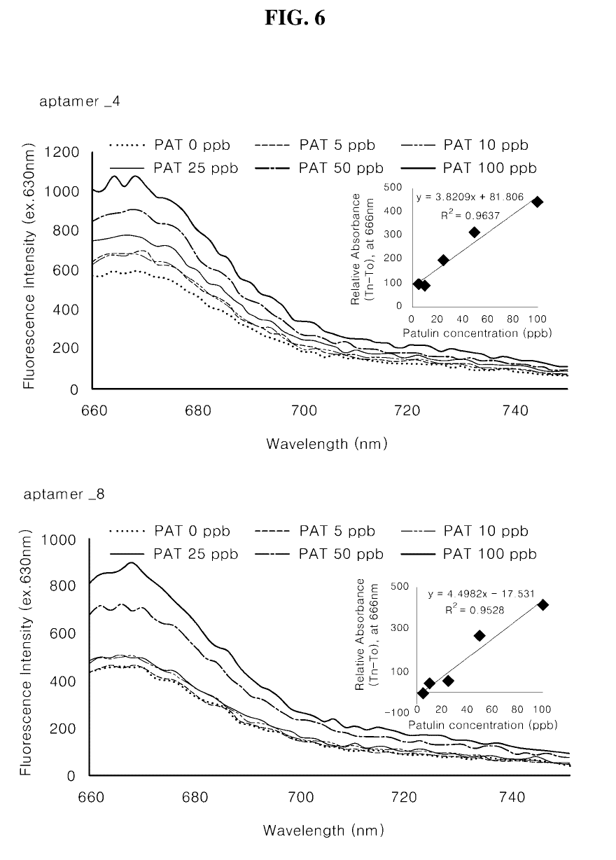

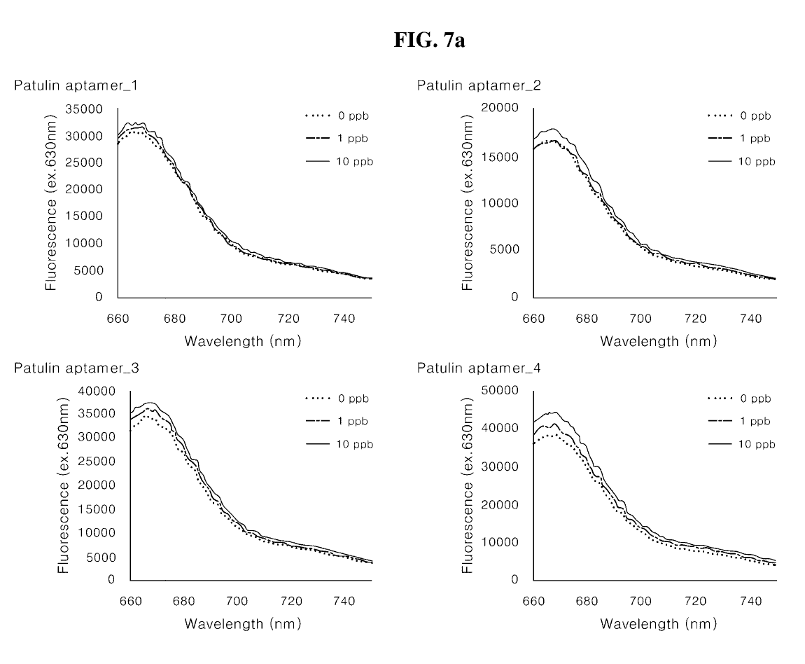

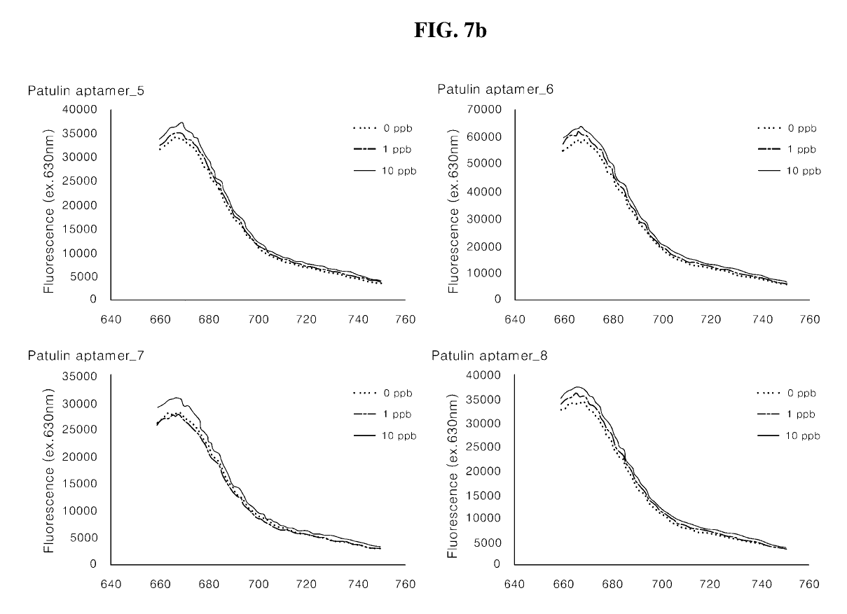

Using the aptamer sequences specific for detecting patulin developed by the above-mentioned method, detection of patulin was performed. In order to identify whether detection was correctly preformed, binding capacity between graphene oxide and the single-stranded DNA and quenching capacity of graphene oxide was utilized. FIG. 5 and FIG. 6 show results of experiments using SEQ ID NO: 4 patulin-specific aptamer and SEQ ID NO: 8 patulin-specific aptamer. For these experiments, each aptamer was constructed by immobilizing a fluorescent material (Cy5). Graphene oxide and the aptamer were bound, which quenched fluorescence of the aptamer by means of graphene oxide serving as a quencher, to which patulin was added. If patulin and the patulin-specific aptamer are bound, fluorescence of the aptamer is recovered while releasing graphene oxide. FIG. 5 is a graph depicting results in a buffer, and FIG. 6 is a graph depicting results in apple juice. FIG. 7a and FIG. 7b shows graphs representing results of eight aptamers of SEQ ID NO: 1 to SEQ ID NO: 8 in a buffer.

More specific procedures for developing an aptamer for specifically detecting patulin and a method for detecting patulin are as follows.

### Example 1: Synthesis of DNA Library Having Single-Stranded Random Nucleotide

A DNA library having a primer region at both ends required for PCR and a 20-50 bp random sequence (N) in the central part was constructed, as shown in below. DNA library used in the present Example was chemically synthesized by Genotech Inc., Korea.

5′-ATT ATG GCG TAT TGC AGC GTT CTG GTT N(20-50)
ATT AGC TTG TTG GTG AGG TAA CGG CT-3′

### Example 2: Selection of DNA Aptamer Binding to Patulin

1 μM of random DNA library synthesized in Example 1 and 1.2 μM of patulin were introduced to a buffer solution (0.1 M MES pH 6.0) and reacted at room temperature for 30 minutes. 2 mg/ml of graphene oxide was added to the buffer solution, followed by reacting at room temperature for one hour, and then DNA which was not specifically bound to patulin was removed through centrifugation at 14,000 rpm for 20 minutes.

### Example 3: Amplification of DNA Aptamer Capable of Binding to Patulin and Preparation of Single-Stranded DNA

In order to amplify amounts of DNA aptamers capable of binding to patulin obtained in Example 2, polymerase chain reaction (PCR) was performed using known primer regions. Since the final product of polymerase chain reaction is a double-stranded DNA, a phosphorylated primer as set forth in below was constructed in order to convert double-stranded DNA to single-stranded DNA.

Forward primer
5′-ATT ATG GCG TAT TGC AGC GTT CTG GTT-3′
Reverse primer
5′-Phosphate-AGC CGT TAC CTC ACC AAC AAG CT-3′

In order to convert the double-stranded DNA as a product of polymerase chain reaction into single-stranded DNA, an enzyme reaction of lambda exonuclease I was performed. The reaction was carried out at 37° C. for 30 minutes, and then the enzyme was inactivated at 80° C. for 10 minutes. Thereafter, the resultant mass was subjected to electrophoresis to separate a double-stranded DNA and a single-stranded DNA, followed by subjecting to a purification kit (Gel extraction kit), thereby obtaining a single-stranded DNA. The single-stranded DNA was used to sequentially perform Examples 2 and 3 up to round 4.

### Example 4: Removal of DNA Capable of Binding to Apple Juice

DNA which passed through Examples 2 and 3 up to round 4 possessed an ability to specifically bind to patulin. Because detection of patulin is generally performed in apple juice, a counter-SELEX method was performed in order to yield DNA which can bind to patulin but does not bind to floating materials in apple juice. By the above procedure, DNA bound to apple juice was removed. Thereafter, the procedures in Examples 2 and 3 were finally performed one more time to secure DNA specifically bound to only patulin. The resultant DNA was cloned into a TA vector to obtain colonies. The colonies obtained from the cloning process were extracted to yield DNA, which was then sequenced. As a result, eight different patulin-specific aptamer sequences were obtained. Eight sequences thus secured are referred to as SEQ ID NO: 1 to SEQ ID NO: 8, respectively (see Table 1). SEQ ID NO: 1 to SEQ ID NO: 8 in Table 1 comprise a forward primer and a reverse primer in their original aptamer nucleotide sequence.

TABLE 1

| SEQ | |
|---|---|
| ID | |
| NO | Sequence (5′ → 3′) |
| 1 | ATTATGGCGTATTGCAGCGTTCTGGTTCTGTGTGCCCCNATNNN |
| | AGGGATTAGCTTGTTGGTGAGGTAACGGCT |
| 2 | ATTATGGCGTATTGCAGCGTTCTGGTTTGGGGGACAGCAGGCGT |
| | CGAAACATTGCCGATTAGCTTGTTGGTGAGGTAACGGCT |
| 3 | ATTATGGCGTATTGCAGCGTTCTGGTTTCGCTCTCAACCTGCTC |
| | TGTATTAGCTTGTTGGTGAGGTAACGGCT |
| 4 | ATTATGGCGTATTGCAGCGTTCTGGTTGAGCTAGGCACGTGCAN |
| | CCCTAAAANGGGTGATTAGCTTGTTGGTGAGGTAACGGCT |
| 5 | ATTATGGCGTATTGCAGCGTTCTGGTTGACCAGTGTGTGTGCGG |
| | ACGTGCCGGGGGTCATTAGCTTGTTGGTGAGGTAACGGCT |
| 6 | ATTATGGCGTATTGCAGCGTTCTGGTTAGGTAACGGCCAGCTTG |
| | TTGGTGAGGTAACGGCT |
| 7 | ATTATGGCGTATTGCAGCGTTCTGGTTGGTGAGGTAACGGCTAG |
| | CTTGTTGGTGAGGTAACGGCT |
| 8 | ATTATGGCGTATTGCAGCGTTCTGGTTTATGGCGTATTGCAGCT |
| | TGTTGGTGAGGTAACGGCT |

### Example 5: Sequencing of Eight Patulin Aptamers and Analysis of Binding Force

Results of sequencing of eight different patulin-specific DNA aptamers secured in Example 4 are summarized in Table 1. Further, secondary structures of eight patulin-specific aptamers predicted using an m-fold program are depicted in FIGS. 3a to 3h.

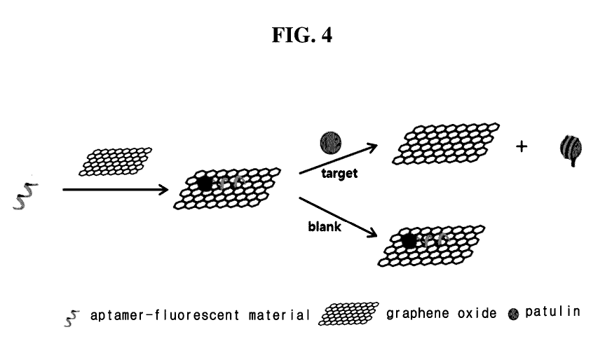

Two of the eight patulin-specific aptamers were selected to analyze their binding force with patulin. As shown in the schematic view of FIG. 4, aptamer-4 (SEQ ID NO: 4) and aptamer-8 (SEQ ID NO: 8) with immobilized graphene oxide and a fluorescent material were bound at room temperature for 30 minutes, followed by adding patulin in each of concentrations and reacting for 30 minutes, thereby measuring rising fluorescence intensity. Namely, a principle employed for this procedure was that the fluorescence signal of fluorescent aptamers quenched by graphene oxide was increased by adding a patulin target having a stronger binding force than graphene oxide, thereby releasing graphene oxide. FIG. 5 shows graphs depicting results of specific binding between a patulin aptamer and patulin in a buffer. In FIG. 5, a blue colored graph in the case of not adding patulin confirmed signals of fluorescent aptamer quenched by binding with graphene oxide. In case of adding patulin in concentrations of 50 ppb and 100 ppb, respectively, it was confirmed that each fluorescence signal was increased. Specifically, the increment width was increasing in proportion to increase in patulin concentration. Namely, it was confirmed that the fluorescence signal was increased depending on the concentration of patulin. FIG. 6 shows graphs depicting results of specific binding between a patulin aptamer and patulin in apple juice as an actual sample. In FIG. 6, it was confirmed that the fluorescence signal of fluorescent aptamers quenched by graphene oxide in an apple juice sample was increased with increasing added patulin. The results showed that the aptamers developed by the present invention could detect 50 μg/ml of patulin in an actual sample.

From the results, it was confirmed that the patulin-specific aptamer sequences secured can specifically bind to patulin and that the patulin-specific aptamer sequences can specifically bind to patulin in apple juice as an actual sample.

FIG. 7a and FIG. 7b shows graphs depicting results of experiments preliminary performed in order to select patulin specific aptamers before the experiments of FIG. 5 and FIG. 6, which revealed that specificity for patulin was found in all of aptamer-1 (SEQ ID NO: 1) to aptamer-8 (SEQ ID NO: 8).

Although some embodiments have been described herein, it should be understood by those skilled in the art that these embodiments are given by way of illustration only, and that various modifications, variations, and alterations can be made without departing from the spirit and scope of the invention. Therefore, the scope of the invention should be limited only by the accompanying claims and equivalents thereof.

What is claimed is:

1. An aptamer for specifically detecting patulin, which has a nucleotide sequence selected from the group consisting of SEQ ID NO: 1 to SEQ ID NO: 8.

2. The aptamer for specifically detecting patulin according to claim 1, further comprising: a forward primer having a nucleotide sequence represented by SEQ ID NO: 9 or a reverse primer having a nucleotide sequence represented by SEQ ID NO: 10.

3. A method for detecting patulin, comprising:
1) immobilizing a fluorescent material or a fluorescent quencher onto an aptamer for specifically detecting patulin, the aptamer having a nucleotide sequence selected from the group consisting of SEQ ID NO: 1 to SEQ ID NO: 8;
2) adding the aptamer to which the fluorescent material or the fluorescent quencher is immobilized to a patulin-containing sample; and
3) selecting an aptamer specifically bound to patulin.

4. The method for detecting patulin according to claim 3, wherein the aptamer further comprises a forward primer having a nucleotide sequence represented by SEQ ID NO: 9 or a reverse primer having a nucleotide sequence represented by SEQ ID NO: 10.

5. The method for detecting patulin according to claim 3, wherein the fluorescent material or the fluorescent quencher is at least one selected from the group consisting of graphene oxide, fluorescein, tetramethylrhodamine, Cy5, Cy3, and Texas Red.

6. A kit for detecting patulin, comprising a patulin detector, wherein the patulin detector detects patulin by reacting a patulin specific aptamer with a patulin-containing sample and selecting an aptamer specifically bound to patulin, and the patulin specific aptamer comprises an immobilized fluorescent material or a fluorescent quencher and has a nucleotide sequence selected from the group consisting of SEQ ID NO: 1 to SEQ ID NO: 8.

7. The kit for detecting patulin according to claim 6, wherein the aptamer further comprises a forward primer having a nucleotide sequence represented by SEQ ID NO: 9 or a reverse primer having a nucleotide sequence represented by SEQ ID NO: 10.

8. The kit for detecting patulin according to claim 6, wherein the fluorescent material or the fluorescent quencher is at least one selected from the group consisting of graphene oxide, fluorescein, tetramethylrhodamine, Cy5, Cy3, and Texas Red.


SEQUENCE LISTING (8 sequences)

SEQ ID NO: 1 - DNA, 74 bp, Artificial
  an aptamer for detecting patulin
attatggcgtattgcagcgttctggttctgtgtgccccnatnnnagggattagcttgttggtgaggtaacggct

SEQ ID NO: 2 - DNA, 83 bp, Artificial
  an aptamer for detecting patulin
attatggcgtattgcagcgttctggtttgggggacagcaggcgtcgaaacattgccgattagcttgttggtgaggtaacggct

SEQ ID NO: 3 - DNA, 73 bp, Artificial
  an aptamer for detecting patulin
attatggcgtattgcagcgttctggtttcgctctcaacctgctctgtattagcttgttggtgaggtaacggct

SEQ ID NO: 4 - DNA, 84 bp, Artificial
  an aptamer for detecting patulin
attatggcgtattgcagcgttctggttgagctaggcacgtgcanccctaaaangggtgattagcttgttggtgaggtaacggct

SEQ ID NO: 5 - DNA, 84 bp, Artificial; identical to SEQ ID NO: 4
  an aptamer for detecting patulin

SEQ ID NO: 6 - DNA, 61 bp, Artificial
  an aptamer for detecting patulin
attatggcgtattgcagcgttctggttaggtaacggccagcttgttggtgaggtaacggct

SEQ ID NO: 7 - DNA, 65 bp, Artificial
  an aptamer for detecting patulin
attatggcgtattgcagcgttctggttggtgaggtaacggctagcttgttggtgaggtaacggct

SEQ ID NO: 8 - DNA, 63 bp, Artificial
  an aptamer for detecting patulin
attatggcgtattgcagcgttctggtttatggcgtattgcagcttgttggtgaggtaacggct